(19) United States
(12) Patent Application Publication
(10) Pub. No.: US 2013/0181141 A1
(43) Pub. Date: Jul. 18, 2013

(54) Ultraviolet Sterilization System

(71) Applicant: Hospira, Inc., Lake Forest, IL (US)

(72) Inventors: Daniel A. Brueck, Highwood, IL (US); Michael D. Lowy, Zion, IL (US); Kevin D. Trupp, Burlington, WI (US); Zhouyao Zhang, Buffalo Grove, IL (US); Randall M. Farmer, Mundelein, IL (US)

(73) Assignee: Hospira, Inc., Lake Forest, IL (US)

(21) Appl. No.: 13/783,249

(22) Filed: Mar. 2, 2013

Related U.S. Application Data
(63) Continuation of application No. 12/905,718, filed on Oct. 15, 2010.
(60) Provisional application No. 61/252,514, filed on Oct. 16, 2009.

(51) Int. Cl. A61L 2/10

(52) U.S. Cl. CPC A61L 2/10
USPC 250/432 R

(57) ABSTRACT

A system for sterilizing packaged products, for example liquid products, with ultraviolet light. The system includes an apparatus for moving product through a treatment zone for a period of time and in a manner sufficient to sterilize the product.

### CROSS REFERENCE TO RELATED APPLICATION

This application is a continuation of U.S. patent application Ser. No. 12/905,718, filed Oct. 15, 2010, which claims the benefit of U.S. Provisional Application Ser. No. 61/252,514 filed Oct. 16, 2009, each of which is incorporated herein by reference in its entirety.

### BACKGROUND OF THE INVENTION

1. Field of the Invention

The invention relates to the sterilization of articles and packaged products using ultraviolet light. In particular, the invention relates to a system and method for the sterilization of certain containers, and the contents thereof, where the containers are constructed of a material that allows for the transmission of light of a preselected wavelength therethrough.

2. Description of Related Art

Large-scale production of sterile medical products requires the use of sterilization systems such as autoclaves, e-beam systems, and gas chambers that use ethyl alcohol sterilization techniques. Autoclaves are pressurized chambers designed to sterilize articles placed therein using steam. Since steam at 134° C. can achieve the same level of sterility in just three minutes that hot air at 160° C. achieves in two hours, autoclaves offer more efficient sterilization than simple heating methods. Also, because autoclaves are pressurized, an autoclave can sterilize a liquid by heating the liquid above its boiling point (at one atmosphere of pressure) without vaporizing the liquid.

The sterilization of medical liquids is often accomplished through the use of large volume autoclaves. These large volume autoclaves allow for the simultaneous sterilization of hundreds of containers containing such medical liquids. Unfortunately, these large volume autoclaves require significant floor space in a manufacturing facility, with the amount of floor space varying based upon the number of units to be sterilized simultaneously in a single autoclave unit and the number of autoclaves required to sterilize the number of units to be sterilized over a given period of time. Construction of such large volume autoclaves requires a significant up-front capital expenditure in terms of both equipment and in the construction of a facility having the amount of floor space to accommodate the autoclaves. In addition, use of these large volume autoclaves requires a significant amount of additional labor due to the fact that units requiring sterilization must be removed from the manufacturing line and placed on racks constructed to withstand the extreme conditions of the autoclave. The racks, once filled, must then be moved into the large volume autoclave. Once the autoclave is filled, it is activated for the required autoclave cycle. Once that cycle has been completed, the racks must be removed from the autoclave and the units removed from the racks, whereupon the units can be placed back into a manufacturing line for further processing and/or packaging. Despite the costs of building and utilizing such systems, autoclave sterilization remains the sterilization technique of choice for large-scale production of medical liquids for parenteral and enteral administration.

Accordingly, the inventors have identified a need in the art for a system and method that offers the ability to sterilize a high volume of articles with minimal disruption to the manufacturing and packaging process and that uses a minimum of manufacturing space.

### SUMMARY OF THE INVENTION

In one aspect, the invention is directed to a sterilization system having a treatment zone, an ultraviolet light source that directs ultraviolet light into the treatment zone, and an apparatus, such as a belt conveyor or movable hanger system, for transporting containers in a substantially continuous manner through the treatment zone and exposing the containers to the ultraviolet light source in a manner and for a period of time sufficient to sterilize the container and the contents of the container. The invention is also directed to a method for sterilizing containers using the system of the invention.

In another aspect, the light source of the system has a housing defined by at least one outer wall having an outer face and inner face. The source also includes a UV lamp positioned within the housing, and a bounded volume of photon-producing gas positioned within the housing. The outer wall includes an area that is substantially transparent to photons produced by the bounded volume of gas, and the area is temperature-controlled through direct contact with a cooling fluid with the inner face.

In a further aspect, the system of the invention measures the intensity of light from a radiant source. The system includes a deep well light sensor positioned to receive light emitted from a single light source without receiving light from other light sources operating nearby. The system may further include a feedback system for controlling the exposure of each container to ultraviolet light. The feedback system may adjust parameters of the system including the speed with which the apparatus moves the containers through the treatment zone, it may adjust of the amount of light emitted by the ultraviolet lamps, or it may adjust both.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

FIG. 1 is an schematic representation of the sterilization system of the invention:

FIG. 2 is an illustration of an embodiment of a hanger system for suspending and transporting fluid-filled containers.

FIG. 3 is an illustration of an embodiment of a hanger system for suspending and transporting fluid-filled containers.

FIG. 4 is an illustration of an embodiment of a hanger system for suspending and transporting fluid-filled containers.

FIGS. 5a and 5b are illustrations of an embodiment of a conveyor for transporting containers.

FIG. 6 is an illustration of a UV light source having a deep well sensor.

### DETAILED DESCRIPTION

The invention is directed to a system and method for sterilizing containers that allow for the transmission of ultraviolet (UV) light therethrough. Although the system and method of the present invention will be described herein in the context of a system and method for sterilizing the contents of such a container, e.g., a liquid pharmaceutical product, it is to be understood that the system and method of the present invention can be used to sterilize empty containers and other products manufactured from materials constructed from materials that are transmissive to UV light. In a particular aspect of the present invention, the system is used to sterilize liquid-filled containers in a continuous process. Such containers include pharmaceutical liquids that require terminal sterilization, for example a “parenteral pharmaceutical product” for human or animal use that is administered in an intravenous or injectable manner. A “parenteral pharmaceutical product” includes, without limitation, injectable products, intravenous products, water for injection, intravenous or injectable nutritional products, irrigation solutions, or the like. Container for other products, e.g., certain liquid food products, may also be candidates for sterilization by the system of the invention.

In general, the system includes a UV light source and an apparatus for moving the containers into the proximity of the source for a period of time sufficient to kill pathogens that may be present in the container. In various embodiments, the system and method are associated with the packaged-product manufacturing process so that after the product is packaged in its primary UV light transmissive container, it can be moved in a continuous manner into the proximity of the light source and then directly to a finishing line for labeling and/or further packaging.

Materials used in the construction of a container to be sterilized in accordance with the present invention should be sufficiently transmissive of ultraviolet light such that the contents of the containers can be sterilized by the wavelengths of ultraviolet energy emitted by UV light sources. For example, certain glass and certain plastic materials are useful in the process of the invention, including PVC-free and DEHP-free materials known for use in the medical applications. An example of such a material is a flex container marketed by Hospira, Inc. (Lake Forest, Ill., USA) under the trademark VisIV®. VisIV® flex containers are made of a 100% PVC-free and DEHP-free, multilayer polyolefin film. However, it will be appreciated that other materials will provide the necessary degree of transmissiveness to UV light, thus enabling them to be used in connection with the present invention.

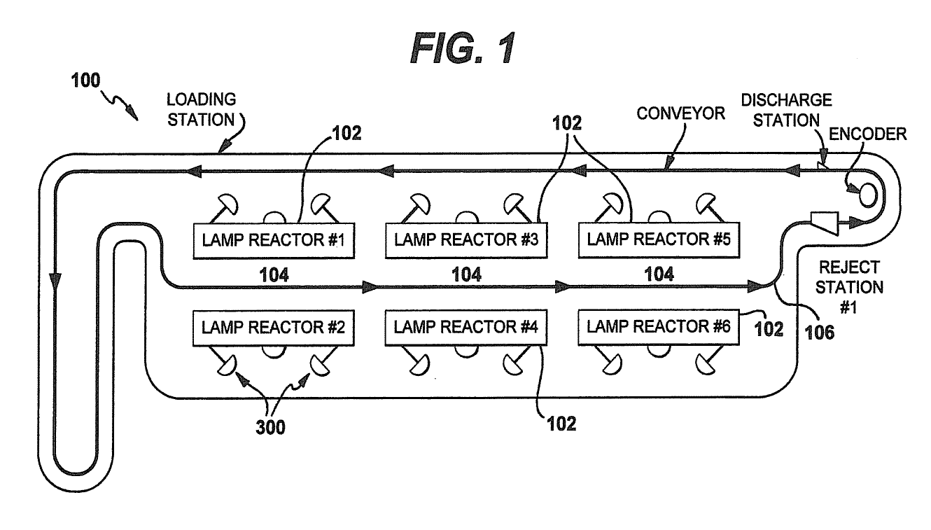

FIG. 1 provides an exemplary illustration of a light sterilization system 100 made in accordance to the principles of the present invention. The system 100 employs one or more light sources 102 for creating a treatment zone 104. The number of light sources 102 used in connection with the system and method of the invention will be dependent upon, among other things, (1) the amount of UV light energy required to sterilize a container (and its contents), (2) the amount of UV light energy emitted by each light source 102, and (3) the speed at which the containers pass through treatment zone 104 (i.e., the time that the container is exposed to UV light energy from light sources 102). Accordingly, while FIG. 1 shows six light sources 102 positioned in spaced relation with respect to each other in order to define the treatment zone 104, as few as one light source may be employed under the appropriate conditions.

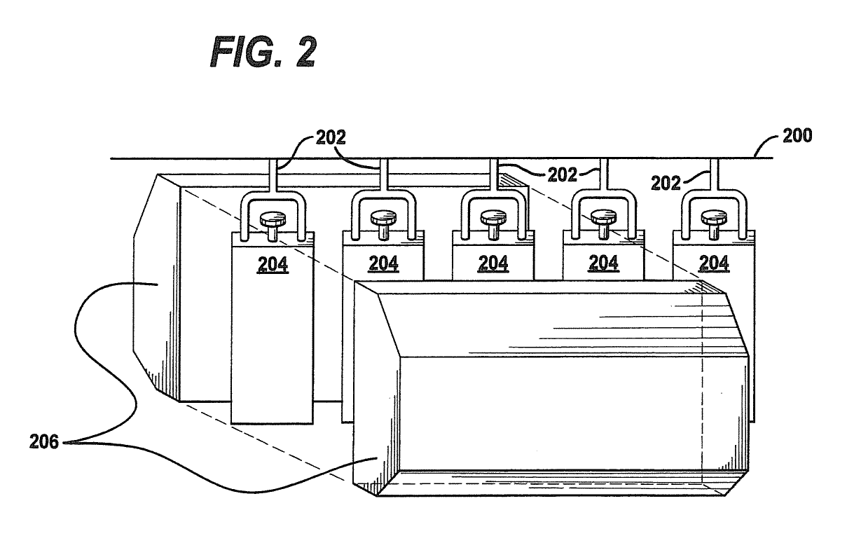

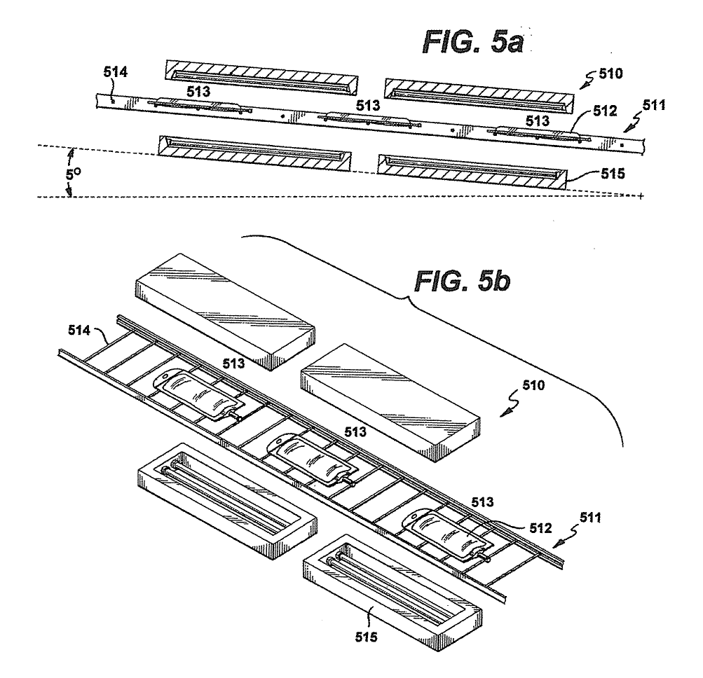

In the exemplary embodiment of FIG. 1, the system uses a transport apparatus 106 for transporting the containers through the treatment zone 104. The transport apparatus 106 should be constructed to allow substantially uninterrupted UV light energy from the source to reach the containers and the contents thereof. As depicted in FIGS. 2-4, the transport apparatus can be constructed to move containers through the treatment zone in a substantially vertical (e.g., hanging) orientation. As depicted in FIG. 5, the transport apparatus can be constructed to move containers through the treatment zone while the containers are supported on a conveyor belt, which may be substantially horizontal or which may be tilted relative to the horizontal, as discussed in further detail below.

In one embodiment (not shown), the treatment zone includes a UV light source on only one side of the container to be treated, e.g., directly above the container or directly below the container, when the transport apparatus is a conveyor belt type system. In another embodiment, the treatment zone includes light sources positioned on at least two sides of the container to be treated, e.g., both above and below the container being transported by the transport apparatus.

Most flexible containers for parenteral solutions contain printed labeling on the primary container in order to identify the contents of the container and provide safety information. It will be appreciated that successful use of the system and method of the invention requires that the printed labeling does not block the UV light radiation emitted by light sources to such an extent that the amount of UV light radiation required to sterilize the container and its contents is not achieved.

The container, when moving through the treatment zone on a transport apparatus, is preferably oriented such that its broadest dimension is oriented substantially perpendicularly to the direction of UV light emitted by light sources. For example, where the container to be treated is an IV flex container and where the light sources are positioned above and/or below the container to be treated, the transport apparatus is preferably constructed such that it presents the container within the treatment zone in a horizontal orientation, i.e., such that the broadest dimension of the flex container is disposed substantially horizontally (e.g., the container lies flat) while the light is being emitted in a substantially vertical direction. In this orientation, it will be appreciated that the width and length of the flex container are substantially greater then the thickness of the flex container, thus the length and width of the flex container are oriented substantially perpendicularly to the direction of the UV light imparted on the container.

In the embodiment of the present invention depicted in FIGS. 5a and 5b, the transport apparatus is a conveyor belt-type of transport constructed such that it allows a substantial amount of UV light radiation to pass through it in order to sterilize a container being transported thereon. For example, the transport apparatus can be a “conveyor belt” constructed of a UV light energy transmissive material, or it can be constructed of a series of filaments or “wires” having a relatively small gauge in order to allow for the transmission of UV light energy to the container through the spaces between the filaments. These filaments can themselves be constructed of a UV energy transmissive material. It has been determined, however, that non-transmissive filaments (e.g., metal wire or mesh) can be used in connection with the present invention so long as they allow for the transmission of sufficient UV light energy to the container and the container's contents. When wires or mesh are used, they should be sufficiently taut and supportive in order to prevent sagging in the supporting plane for the containers. A variety of configurations are available, including wire meshes, that provide the required support while being sufficiently transmissive.

Alternatively, the transport apparatus can be configured as a plurality of wells, baskets or trays constructed to present the container in a desired orientation. The wells, baskets or trays can be constructed of a material that is transmissive to UV light energy per se, or can be constructed from a plurality of filaments or wires that allow for the transmission of UV light energy therethrough.

In cases where the treatment zone includes light sources oriented such that they emit UV light in a substantially horizontal direction, and where such light sources are positioned on either side of the container to be sterilized, the container is preferably presented to the treatment zone such that its broadest dimension is oriented in a vertical plane. That is, the containers are preferably transported through the treatment zone by a transport apparatus that orients the packages vertically. In the case of a flex container, this means that the length and width of the flex container are oriented in a substantially vertical plane while the thickness of the flex container is oriented in a substantially horizontal plane, i.e., parallel to the plane in which the light sources are emitting light energy. This can be achieved by using a transport apparatus that includes clips or hanger elements that will releasably attach to the container to be sterilized. Clips can take a variety of known configurations so long as they do not damage the container and so long as they do not prevent the container from being properly sterilized, i.e., they do not block significant amounts of UV light energy from reaching the container and the contents of the container. Hangers can take a variety of known configurations, including, for example, a recess configured to receive a port associated with a flex container such that the port can be inserted into the hanger's recess in order to hang the flex container from the port.

UV light sources useful in connection with the system and method of the present invention can take a variety of forms. In one embodiment, each light source is constructed to deliver substantially monochromatic light to each container at radiance levels of about 200 mW/cm² to 600 mW/cm². In this embodiment, the UV light source has a relatively narrow emission spectra (i.e., produces substantially monochromatic light), for example, within a wavelength range of approximately 282 nm±5 nm. This range of wavelength interacts with DNA, and when applied at the appropriate dosage, destroys a spectrum of pathogens while leaving the treated product unaffected. In other embodiments, the light may be generated and delivered at other discrete wavelengths, e.g., wavelengths of 193 nm; 207 nm; 222 nm; 248 nm; 254 nm; 308 nm; 354 nm and 361 nm. The light wavelength may be controlled to, for example, ±1 nm, ±2 nm, ±3 nm, ±5 nm, or ±5 nm. For example, the monochromatic UV light wavelength may be controlled to a selectable bandwidth to optimize container penetration and microbial kill. In general, the system preferably administers a dosage of UV light energy necessary to achieve a six log reduction of pathogens within the container and/or a sterility assurance level (SAL) of 10⁻⁶, which is an expression of probability reflecting that, following treatment, one package in one million might be nonsterile.

In one embodiment, the light source 102 is a reactor lamp produced by Triton Thalassic Technologies, Inc. (Ridgefield, Conn.). Additional details regarding these Triton light sources are set forth in U.S. Pat. Nos. 7,381,976; 7,282,358; 7,217,936; 7,057,189; 6,201,355; 5,834,784; and 5,626,768, each of which is incorporated herein by reference in its entirety.

The number and spatial orientation of the light sources will vary depending upon a number of factors including the size of the container to be sterilized and the limits of sterilization to be achieved. e.g., US 2010/007492, which is incorporated herein by reference in its entirety. The number of light sources may be increased to ensure that containers are resident in the treatment zone for a period of time sufficient to ensure sterilization of the contents of containers. Thus, if the conveyor system is intended to operate at a rate of speed such that a single container does not reside in the treatment zone for the minimum period of time required to ensure sterilization of the contents of the container, additional light sources may be positioned along the pathway of the transport apparatus to provide a length of the treatment zone that ensures sterilization. In addition, the speed of the conveyor can be adjusted to ensure that the containers remain in the treatment zone for a period of time sufficient to achieve sterilization.

The amount of light to which containers are exposed may vary throughout the treatment zone depending upon the characteristics of the ultraviolet light sources. For example, the intensity of the ultraviolet light emitted by a single light source may be greatest at the centerline of the light source, with the amount of light decreasing in proportion to the distance from the centerline of the light source. To ensure that containers and their contents are exposed to sufficient ultraviolet light for desired sterilization, the relative orientations of the light sources may be adjusted such that the entireties of the containers are exposed to the required amount of ultraviolet light. By way of example, the light sources can be oriented such that one light source is positioned on a first side of the transport apparatus while two light sources are positioned on the opposite side, with the two light sources being positioned such that their respective centerlines are above and below, i.e., offset relative to, the centerline of the light source on the opposite side of the apparatus. The light sources can be positioned at a distance from the containers at distance that maximizes the total amount of light energy to reach the entire container.

When the transport system is a belt-type conveyor system, and where the containers are supported on the belt as they are transported through the treatment zone, the belt may be oriented so that it is slightly offset relative to horizontal, i.e., the conveyor is pitched so that one end of the container is oriented higher than the other end of the container as it is moved by the conveyor through the treatment zone. For flexible containers that contain liquids and that have an air space, this non-horizontal orientation will tend to cause air within the container to move to the higher end of the container as it is subject to UV light in the treatment zone. This orientation maximizes the amount of liquid that is exposed to UV light that passes directly through the container wall and into the liquid instead of the light also having to pass through air space(s) within the container before entering the liquid. This orientation also ensures a more uniform exposure of the liquid to the light. In addition, this embodiment helps to avoid other air bubbles, large or small, or air pockets within the container. It has been found that the conveyor may be pitched as little as about 5-10 degrees relative to horizontal in order to ensure that the air in the container is moved to the most highly elevated position within the container. It will be appreciated that greater pitch angles can be used. However, the pitch should not be so great as to cause the containers to slip on the belt so that one container contacts another or falls off the belt. However, it is possible to add additional stabilizing structures onto the belt in order to prevent such container slippage if it is deemed desirable to utilize such a high pitch.

In a further aspect of a non-horizontal conveyor system of the invention, the conveyer system can be constructed such that it moves through the treatment zone in a substantially horizontal orientation while simultaneously tilting containers such that one side edge of the container is higher than the other. It will be appreciated that this can be achieved by providing pitched structures on the conveyor system. For example, individual “platforms” constructed from UV light transmissive materials and/or from wire filaments or mesh can be provided on the surface of the conveyor system in order to provide the desired pitch to the container while simultaneously allowing the conveyor system to move horizontally through the treatment zone. Similar to the pitched embodiment of the conveyer, this embodiment allows any air in a container to accumulate in the most elevated portion of the container. If the conveyor is tilted within the treatment zone, the angle of the tilt should not be so great so that it causes the containers to slip off the conveyor or come into contact with other containers on the conveyor system. It will be appreciated that any additional structures provided on the conveyor system, e.g., guards, rails or holders, be constructed such that they block as little UV light as possible, thereby facilitating sterilization of containers passing through the treatment zone and minimizing the amount of UV light required to achieve the desired sterilization effect.

In an even further aspect of a non-horizontal conveyor system, the conveyor changes pitches within the treatment zone so that the air pocket within the container moves from one end of the container to the other as the container passes through the treatment zone. For example, the elevation of the conveyor system may have a crown in the middle of the treatment zone such the conveyor is inclined through a first portion of the treatment zone and declined in a second portion of the treatment zone. Alternatively, the conveyor can be constructed to decline in the first portion of the treatment zone and incline in the second portion of the treatment zone.

The orientation and intensity of the light sources within the treatment zone must consider the container size. Light intensity directly in front of a light source is usually higher than at the periphery. Containers to be sterilized in accordance with the system and method of the present invention are preferably positioned relatively close to the light sources when the containers are in the treatment zone, thereby minimizing the amount of light attenuation that occurs before the containers are subjected to the UV light from the light sources. The precise spacing of the light sources in the treatment zone will need to be varied based upon the size of containers passing through the treatment zone.

It will be appreciated that flex containers containing medical fluids may include inlet and/or outlet ports for accessing the liquid therein. Because these ports are generally made of plastic that is thicker and less transmissive than the other material of the container, it may be necessary to sterilize these ports separately before they are incorporated into the container. Such sterilization can be achieved through a variety of known techniques, including e-beam sterilization.

It should be understood that while the containers sterilized in accordance with the present invention may be completely liquid-filled, the containers may also be partially filled, without departing from the scope of the invention. Indeed, flexible containers for parenteral solutions typically contain some air space. The present invention can also be used to sterilize empty containers before they are filled.

In the embodiment depicted in FIG. 2, the transport apparatus is a hanger system including a moving wire with the packaged products suspended from the wire as they are transported through a treatment zone of one or more UV light sources directed at the package from one or several sides of the package. The transport system 200 employs hangers 202 to suspend/retain the containers 204. While retained by the hangers 202, the containers 204 are oriented in a substantially vertical position. In order to maximize the amount of light to which containers 204 are exposed, the width of the containers 204 can be oriented in the direction of travel through the treatment zone in between light sources 206. Further, the containers 204 are oriented such that the inlet ports are located at the top of each container 204. Since containers 204 may be flexible, the liquid may be unevenly distributed when containers are oriented vertically, with a greater percentage of the liquid volume being located in the bottom portion of the container. Accordingly, the light source and the residence time of the package in the treatment zone must accommodate a package that has a non-uniform configuration. For example, it may be necessary to supply greater UV light energy to the lower portions of the containers, either through the use of an increased number of light sources 206 or through the use of light sources 206 that have a greater output of UV light.

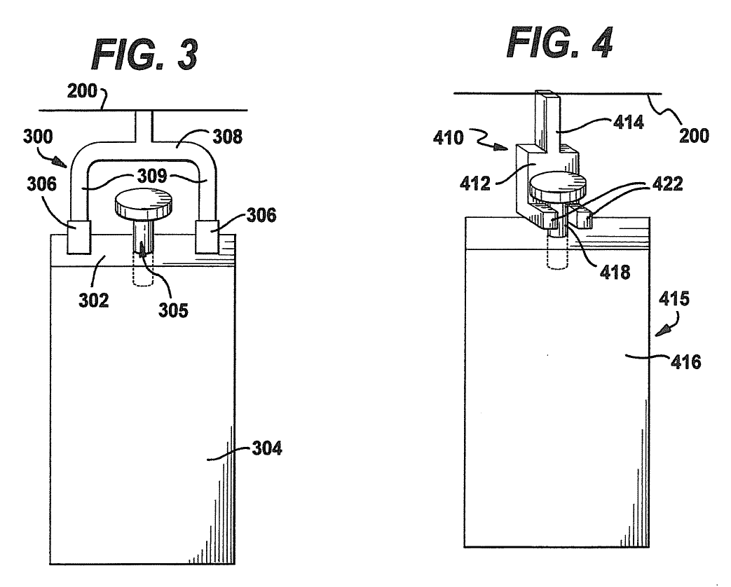

FIG. 3 is a schematic illustration of an alternative hanger 300 for suspending a liquid-filled container. Hanger 300 includes two or more fasteners 306 and an arm 308 having branches 309, and may be part of a hanging system for the transport apparatus 200. Hanger 300 is designed to be used with containers including a sealed section 302, a body 304 and an inlet port 305. The sealed section 302 is air-tight such that no liquid can pass from the liquid-filled body 304 or from the inlet port 305 into the sealed section 302.

The fasteners 306 are designed to attach to the sealed section 302 of the container. Preferably, when attached to the sealed section 302 of the container, fasteners 306 are positioned so that they do not substantially block ultraviolet light from penetrating the liquid-filled body 304 of the container or the inlet port 305. The fasteners 306 may take various forms, such as clips or hooks (if holes are provided in the sealed section 302 for the hooks), among others. In the depicted embodiment, the fasteners 306 are attached to the transport apparatus 200 with a single arm 308 with branches 309, although an alternative embodiment may use an individual arm for each fastener.

FIG. 4 is a schematic illustration of another alternative hanger 410 for suspending a liquid-filled container. Hanger 410 includes a support 412 and an arm 414, and may be part of a hanging system for the transport apparatus 200. The support 412 is configured to suspend the container 415, which includes a liquid-filled body 416 and an inlet port 418. Support 412 includes a notch created by brackets 422 that extend from the support 412. The inlet port 418 is inserted into the notch so that hanger 410 may support the container. While this support arrangement may obstruct some ultraviolet light from penetrating the inlet port 418, the container 415 may be filled in such a manner that the non-exposed portion of the inlet port 418 does not contain a substantial volume of liquid. Further, to assist in sterilization of liquids within the inlet port 418, the container 415 may incorporate a pre-sterilized inlet port.

FIGS. 5a and 5b show a sterilization system 510 that includes a conveyor belt system 511 for supporting the flexible liquid containers 512 as they pass through the treatment zone 513. The conveyor 511 is constructed of a series of thin wires 514 (e.g., aluminum wire) that is sufficiently taut to minimizing sagging under the weight of the supported container. UV light sources 515 are positioned above and below the conveyor 511 to supply the requisite UV light to sterilize the containers and their contents as they pass through the treatment zone. FIG. 5a shows that the conveyor is pitched relative to horizontal at approximately five degrees so that the liquid contents of each container accumulate at one end of the container.

Turning back to the light source, an ultraviolet reactor lamp generally includes a housing, a light source positioned within the housing; and electrical connections that supply power to the light source. The housing contains a bounded volume of photon-producing gas. An outer wall of the housing has a window that is substantially transparent to photons produced by the bounded volume of gas. Preferably the window is constructed of transparent quartz.

Further, a cooling fluid is applied to the inner face of the substantially transparent window of the outer wall in order to control the temperature of the outer wall. To facilitate this temperature control, the light source may include a bounded region that is adapted to receive the cooling fluid. In an exemplary embodiment, the cooling liquid passes in direct contact with the inner face of the outer wall and then enters the bounded region of the light source to provide cooling to the light source as well. In various embodiments, the cooling liquid is USP water for injection that may contain sodium chloride or sodium carbonate. In other embodiments, the cooling water is tap water that contains an appropriate amount of minerals to obtain a range of conductivity of between about 200 to about 500 mSiemens, or more particularly between about 300 to about 400 mSiemens. Tap water can be diluted with water for injection or distilled water to provide the appropriate conductivity or as required by the electrical service delivery to the lamp. Depending upon the source of the tap water, the dilution generally can be within the ranges of 1:2 to 2:1 (tap water:distilled water), but this range can vary widely depending upon the source of the tap water. As would be recognized by one of skill in the art, the water, and the mineral contents thereof, should not be corrosive to any aspect of the light source or cooling system.

In one embodiment, the light sources are parallel with the conveyor system. Therefore, when the conveyor is inclined, the light sources are also inclined. (See FIG. 5a). This allows and air bubbles in the cooling fluid of the light sources to collect at one end of the housing so that the bubbles are not in the path of the light. By reducing or eliminating the possibility of an air bubbles interfering with the radiation of UV energy from the source, a more predictable level of radiation can be achieved and the amount of power used in the system can be reduced. Even if the conveyer system is horizontal, the light source may be inclined to the extent necessary to reduce or eliminate air bubbles in front of the window on the sources. Of course, sources should not be inclined so much as to cause one end of the light to be too far from the conveyor such that it would substantially affect the intensity of light delivered to the containers.

UV light intensity in the treatment zone is a function of the energy produced from each lamp. In one embodiment, UV sensors and an adjustable power supply are installed on each UV lamp. A controller can use a closed loop algorithm to maintain the light intensity by controlling the output of the power supply to the UV lamp. Accordingly, the system is equipped with intensity sensors to monitor the UV light and a controller to adjust the power supply to the lamp based upon intensity of the light measured by the sensors. UV light sensors are available from a variety of sources including sglux GmbH, Berlin, Germany.

In particular aspects, the UV sterilizer system has multiple lamps that focus the UV light into the treatment zone. In order to properly control each lamp individually to a specific intensity set point, UV light from adjacent lamps should not be sensed by the light sensor of the lamp being monitored. Accordingly, in one embodiment, the housing of a UV light source includes a deep well sensor to eliminate light from all the adjacent lamps. In one embodiment, the sensor in the deep well is from the UV-Air® sensor series from sglux GmbH (e.g., UV_Air_ABC_AMP4-20 mA_cable).

As shown in FIGS. and 1 and 6, lamp geometry may be used to prevent light from one lamps in the system from reaching the sensor of any other lamp. A UV intensity sensor 300 is placed in a deep well 302 where the optical path intersects the lamp being monitored and uses the endplate 304 of the inner wall (interior wall not shown in the figure) of the housing to shield the adjacent light.

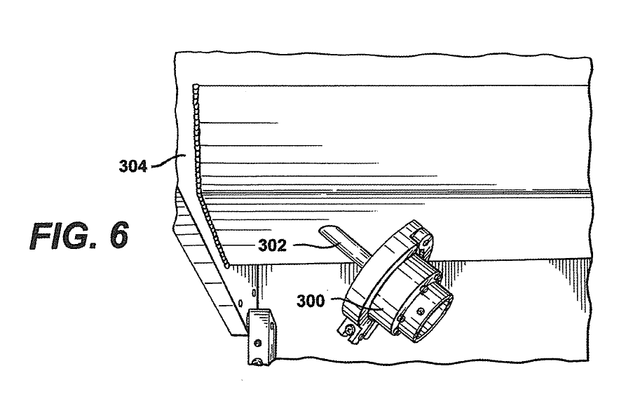

FIGS. 1 and 6 illustrate the deep well mounted at an angle focusing on the endplate of the lamp. The end plates effectively shield the intensity sensor from UV light emitted by all other lamps in the system. Stray UV light that strikes the well at an angle bounces a few times and is nearly eliminated before it reaches the intensity sensor. The depth of the well 302 is such that stray UV light undergoes multiple bounces on non-reflective surfaces before being incident on the intensity sensors. The stray UV light is reduced at every bounce. Only the light from the lamp being monitored has a direct optical path to the intensity monitor.

A UV-exposure control system may employ a feedback system in order to control, and preferably keep substantially constant, the amount of UV light to which each container is exposed while passing through the treatment zone. The feedback system may be configured to adjust the speed of the transport apparatus or may be configured to adjust AC voltage supplied to one or more of the ultraviolet lamps in order to increase or decrease the amount of UV light produced by each lamp.

In one embodiment, the light intensity at the deep well sensor is measured and compared to the desired setpoint. If the value for the UV intensity measured by the sensor decreases from the setpoint, then additional power can be supplied to the lamp at predetermined time intervals, e.g., 50 watts added to the power set point every twenty (20) seconds until it reaches maximum allowable higher adjustment +1.0 kW. If the value for the UV intensity increases from the setpoint, power to the lamp is decreased, e.g., 50 watts subtracted from the power setpoint every twenty (20) seconds until it reaches maximum allowable lower adjustment −0.5 kW.

Typical organisms that may be eradicated by the system of the invention include, for example, Bacillus pumilus (spore former), Candida albican (yeast), lipid and non-lipid virus, Clostridium sporogenes (anaerobic spore former), Alicyclobacillus, Staphylococcus aureus (vegetative Gram positive), Pseudomonas aeruginosa (vegetative Gram negative), Aspergillus niger (filamentous fungi), Mycobacterium terrae, Porcine Parvo Virus (PPV and B19), Lysteria, Salmonella, B. atrophaeus, M luteus and S. maltophilia. In exemplary embodiments, a defined UV intensity range may be established for each container/product grouping (e.g., container size, fill-volume, etc.). The control system may automatically adjust the power to each UV light source to maintain each defined UV intensity range for each container/product grouping. The treatment parameters (e.g., UV intensity range) are generally selected based on the treatment for a single organism, or for multiple organisms.

In another aspect, the transport apparatus moves the containers through the treatment zone in a substantially continuous manner. This allows the system to be part of the manufacturing process, and does not require batch type sterilization typically used in high-volume sterilization methods. The substantially continuous movement of the transport apparatus includes embodiments having start-stop intervals within the treatment zone, as long as the movement of the packages through the zone occurs sequentially, and without the need to accumulate a number of packages for batch sterilization.

The transport system may be readily incorporated into an in-line (i.e., a continuous or semi-continuous) process. For example, the transport system may be incorporated immediately downstream of the container filling operation, wherein the transport system delivers the recently filled container to the treatment zone immediately after filling. Sterilization can be accomplished within a short time, e.g., one minute or less, of filling. In exemplary embodiments, the transport system is adapted to transport the containers through the treatment zone at a rate up to about 100 containers per minute or more, depending on the intensity of the UV light, the length of the treatment zone, and the speed of the conveyor. The speed of the conveyor can be increased without increasing the intensity of UV light from each light source by increasing the length of the treatment zone, i.e., adding additional light sources adjacent to the treatment zone path at positions upstream or downstream from the original treatment zone, thereby allowing the containers to remain in the treatment zone for the sufficient amount of time.

When a transport system uses a conveyor support to transport containers through the treatment zone, the system may include an apparatus for uniformly presenting the containers to (or placing the containers on) the conveyor to ensure that each container is transported through the treatment zone in a consistent and repeatable manner. This is preferable when the conveyor system is accepting containers directly from the filling line and prevents containers from entering the treatment zone in an orientation that would prevent a container from receiving the amount of UV radiation necessary to achieve the desired sterilization effect.

In one embodiment, the apparatus for presenting the containers to the conveyer may include a system for dropping or placing the containers on the conveyer accompanied by rails or guides which ensure that the containers are positioned in a desired orientation relative to the conveyor and relative to the light sources 102 in treatment zone 104. In another embodiment, the apparatus includes a holding station positioned above the conveyer that accepts the container from the filling line. At timed intervals, the station drops the container on the conveyor in a uniform and proper orientation. One or more holding stations can be used serially, each of which simultaneously or sequentially drops the containers onto the conveyor, the timing of which should prevent one container from being dropped on top of another container.

In a further embodiment, the transport apparatus includes a vision system to detect whether the containers are properly oriented on the conveyor before the containers enter the treatment zone. The vision system can also detect whether the containers are overlapping or whether there may be other irregularities in the containers or the position of the containers on the conveyor. If any irregularity in a container, or its position or orientation, is detected, the system can reject the container either manually or electronically, or stop the process to allow the operator to correct the process.

Although various specific embodiments of the present invention have been described herein, it is to be understood that the invention is not limited to those precise embodiments and that various changes or modifications can be made by one skilled in the art without departing from the scope and spirit of the invention.

We claim:

1. A process for sterilizing contents of an ultraviolet (UV) light transmissive container, comprising:
(a) providing a UV light transmissive container having contents to be sterilized;
(b) loading the container on a transport apparatus comprising a belt conveyor having a first portion constructed such that the container, when loaded on the first portion of belt conveyor, is oriented at a first angle relative to horizontal;
(c) using the transport apparatus to transport the container through a treatment zone in which the container is exposed to ultraviolet (UV) light for a period of time sufficient to sterilize the contents of the container

2. The process of claim 1 wherein the belt conveyor is constructed from filaments or a mesh.

3. The process of claim 1 wherein the belt conveyor is constructed from a material that is substantially transmissive to UV light.

4. The process of claim 1 wherein the belt conveyor comprises at least one of a group consisting of wells, baskets, and trays.

5. The process of claim 1 wherein the first angle is in a range of about 5° to about 10°.

6. The process of claim 1, wherein the belt conveyor has a second portion constructed such that the container, when loaded on the second portion of belt conveyor, is oriented at a second angle relative to horizontal;

7. The process of claim 1 wherein the transport apparatus further comprises a pitched structure constructed to support the container thereon, the pitched structure being supported by the belt conveyor, whereby when the container is supported on the pitched structure the container is oriented at the first angle relative to horizontal.

8. The process of claim 1 further comprising providing a system configured to measure an intensity of UV light within the treatment zone and to control an operating speed of the transport apparatus and/or the intensity of UV light in the treatment zone based upon the measured intensity of UV light.

9. The process of claim 8, wherein the system comprises a UV light sensor positioned within a well.
